(19) United States
(12) Patent Application Publication
(10) Pub. No.: US 2025/0035606 A1
(43) Pub. Date: Jan. 30, 2025

(54) Water Analysis with Ultra-Thin Solid State Nanopores

(71) Applicant: Goeppert, LLC, Philadelphia, PA (US)

(72) Inventors: David Niedzwiecki, Philadelphia, PA (US); Zehui Xia, Philadelphia, PA (US)

(21) Appl. No.: 18/220,701

(22) Filed: Jul. 11, 2023

(51) Int. Cl. G01N 33/18; B82Y 15/00; B82Y 30/00

(52) U.S. Cl. CPC G01N 33/1826; B82Y 15/00; B82Y 30/00

(57) ABSTRACT

Ultra-low noise nanopore chips with ultra-high signal are produced at the wafer scale by combining fused-silica chips that have ultra-low capacitance (which dominates signal noise at high bandwidth) with local ultra-thin regions. The method uses electron-beam lithography (EBL) and hydrofluoric acid (HF) etching is applied to the fused-silica chips in a manner designed to avoid labor intense thinning techniques and to ensure reproducible thickness of the membrane while maintaining the robust character of the resulting nanopore chips. The resulting nanopore chips are used to detect small organic analytes (˜1 nm), such as diethyl phthalate (DEP), that are desirable to measure for water monitoring applications such as in the water system aboard the International Space Station (ISS) and other spacecraft.

### GOVERNMENT INTEREST

The subject matter described herein was developed with funding received under NASA Contract Number 80NSSC21C0368. NASA may have certain rights in the subject matter described herein.

### TECHNICAL FIELD

Examples set forth herein generally relate to methods using a solid-state nanopore chip to analyze water to detect small inorganic analytes (e.g., <1 nm diameter) and the wafer-scale manufacture of ultra-thin low-noise membranes for such solid-state nanopore chips.

### BACKGROUND

Solid-state nanopores are excellent candidates for robust and ultra-fast single-molecule detection with sub-nm precision, making them the ideal sensors for low-concentration sample detection. A solid-state nanopore is a nanoscale or larger hole (from a few nm to hundreds of nm) drilled into an inorganic-made plate, e.g., silicon nitride (SiNₓ). SiNₓ pores can withstand high temperatures and be sterilized to minimize contamination. In the early 1990s, nanopores were envisioned in the context of protein pores (alpha-hemolysin) by Kasianowicz et al., who used them to analyze individual polynucleotides. SiNₓ nanopores were first fabricated by using electron and ion beam drilling. They are useful in biomolecular detection, and promising for new diagnostic and filtering devices. The simplest nanopore instrument contains an electrolytic solution separated by the pore. When voltage is applied, the ionic current flowing through the pore is measured (on the order of 0.1 nA to 1 nA). The modulation of current caused by the passage of a molecule through the pore reflects the physical and chemical properties (e.g., size, shape, charge), creating electrical “fingerprints” that may form a basis for quantitative detection. Nanopore diameters, materials, and properties can be fine-tuned using a range of techniques from electron irradiation to electroporation and down to single-atom-thin pores in a multitude of materials and sub-nm pore diameters. SiNₓ pores can distinguish monomer and dimer proteins of only 33 amino acids long, analyze antibiotic/RNA complexes, identify the percentage of hydroxymethyl cytosine (hmC) nucleotide within a larger DNA strand, and distinguish between different DNA homopolymers and individual DNA nucleotides.

A solid-state nanopore based single-molecule detection instrument has been developed by the present inventors for the search of life in outer space, featured on the cover of the Review of Scientific Instruments (March 2020). This portable, cm-scale instrument relies on fast electronics (up to 200 kHz) and thin (5-20 nm) solid-state nanopores in low-capacitance glass, and the platform works with a variety of sample formats (solid, liquid, etc.), dissolved in salt solutions for measurements. This platform has been used to detect and characterize proteins, mRNA, and DNA in artificial seawater and Mars analog soils, and small pharmaceutical molecules (˜1 nm) in aqueous samples.

FIG. 1 shows schematics of a solid-state nanopore chip 10 developed by the present inventors for measuring ionic current flow through a solid-state hole (“nanopore”). As shown in FIG. 1A, in the absence of the particle in the electrolytic solution separated by the pore 12, the current signal is constant with a root-mean-square (rms) noise, Iᵣₘₛ, that mostly depends on the chip capacitance and instrument specifications. As shown in FIG. 1B, the modulation of the current caused by the passage of the molecule 14 through the pore 12 creates a current pulse or “fingerprint” that forms a basis for quantitative detection. FIG. 1C illustrates a Transmission Electron Microscope (TEM) image of a 20 nm-thickness SiNₓ nanopore. FIG. 1D illustrates a sample nanopore detection instrument disclosed by the present inventors including the low-noise nanopore chip (e.g., 5×5×0.2 mm³) 10 inserted into a fluidic cell 16. The fluidic cell (e.g., 25×15×5 mm³) 16 with microfluidic channels forming reservoirs in contact with the nanopore chip 10 is inserted into the portable nanopore reader (e.g., 10×4.5×2 cm³) 18 for detection.

Such solid-state nanopores can act as single-molecule sensors; however, signal-to-noise ratio limits the minimum size of analytes that can be detected. In general, smaller molecules move through the nanopore more quickly, which requires sensing electronics to operate at higher bandwidth, which increases the baseline noise of the system. Smaller molecules also produce less current modulation in the ionic current signal, which means a lower signal for detection. Both of these combine to reduce the signal-to-noise ratio for small molecules.

Chip capacitance is the major contributor to baseline noise at high bandwidth. Therefore, reducing chip capacitance improves noise. The current signal is inversely proportional to membrane thickness. Therefore, reducing chip thickness increases the signal level. Unfortunately, while the signal increases with thinner nanopores, very thin pores are fragile. They become more fragile when they span a large area. Local thinning has been used to minimize breakage. For example, Electron Beam Lithography (EBL) and Reactive Ion Etching (RIE) have been used to locally thin the membrane. The resulting membrane provides a greater current signal while maintaining membrane integrity and low chip capacitance. However, such local thinning techniques require time intensive alignment-to-membrane adjustments which are complicated by the non-flat surfaces produced by strain between nitride and silicon surface. EBL to fused silica is further complicated by surface charging during alignment.

### SUMMARY

Various examples are now described to introduce a selection of concepts in a simplified form that are further described below in the Detailed Description. The Summary is not intended to be used to limit the scope of the claimed subject matter.

Sample configurations described herein relate to a method of identifying unwanted constituents present in water systems. The method has application for testing water aboard a spacecraft. Methods of fabricating a nanopore chip for use in the water monitoring system are also described.

In sample configurations, the methods include a method of sensing an organic analyte (e.g., diethyl phthalate (DEP)) in a fluid (e.g., water) by inserting a solid-state nanopore chip having at least one nanopore of less than 5 nm diameter into a fluidic cell, providing a fluid for testing for the organic analyte, the organic analyte having a diameter of approximately 1 nm or less, and measuring concentration of the organic analyte as the fluid passes through the at least one nanopore of the solid-state nanopore chip by, for example, recording translocation events of organic analytes one at a time using a nanopore reader as the organic analytes translocate through the at least one nanopore. In accordance with the methods, the solid-state nanopore chip comprises at least one silicon nitride (SiNₓ) nanopore having a thickness of approximately or smaller than 5 nm at the at least one SiNₓ nanopore. The at least one nanopore may have a diameter of approximately 2 nm, approximately 1.9 nm, or approximately 1.5 nm.

A water monitoring system for applications such as water monitoring on a spacecraft is also described that is adapted to sense an organic analyte (e.g., diethyl phthalate (DEP)) in water. The water monitoring system includes a fluidic cell that holds water for testing, a solid-state nanopore chip having at least one nanopore of less than 5 nm diameter disposed in the fluidic cell for testing the water for the organic analyte, the organic analyte having a diameter of approximately 1 nm or less, and a nanopore reader that measures concentration of the organic analyte as the water passes through the at least one nanopore of the solid-state nanopore chip by, for example, recording translocation events of organic analytes one at a time as the organic analytes translocate through the at least one nanopore. In sample configurations, the solid-state nanopore chip comprises at least one silicon nitride (SiNₓ) nanopore having a thickness of approximately or smaller than 5 nm at the at least one SiNₓ nanopore. In sample configurations, the at least one nanopore has a diameter of approximately 2 nm, approximately 1.9 nm, or approximately 1.5 nm.

A method of fabricating an ultra-thin (˜<5 nm) solid-state nanopore chip having at least one nanopore of less than 5 nm diameter is further described. The described fabrication method includes coating a fused silicon oxide glass wafer on a first side with a low stress SiNₓ and poly-silicon and removing a top poly-silicon layer from the first side by wet etching with potassium hydroxide (KOH). Small regions and backside alignment marks are patterned using electron-beam lithography (EBL) and thinned to 15 nm or less using trifluoromethane (CHF₃) and O₂ reactive ion etching (RIE). The EBL-patterned regions may be coated with chromium via pressure vapor deposition (PVD). In sample configurations, back side alignment photolithography and RIE is used to remove the SiNₓ opposite the thinned regions, and hydrofluoric acid (HF) etching is used to etch the silicon oxide glass wafer to create SiNₓ windows. The glass wafer with SiNₓ windows is diced into approximately 5×5 nm² chips, and fine HF etching and KOH etching are applied to remove poly-silicon to expose the SiNₓ windows. The chromium is removed by a chromium etchant to create the freely suspended SiNₓ membrane. The fabrication method may further include thinning the SiNₓ membrane to a thickness of as thin as 1.5-2.0 nm using CHF₃ and O₂ RIE.

This summary section is provided to introduce aspects of the inventive subject matter in a simplified form, with further explanation of the inventive subject matter following in the text of the detailed description. The particular combination and order of elements listed in this summary section is not intended to provide limitation to the elements of the claimed subject matter. Rather, it will be understood that this section provides summarized examples of some of the embodiments described in the Detailed Description below.

### BRIEF DESCRIPTION OF THE DRAWINGS

In the drawings, which are not necessarily drawn to scale, like numerals may describe similar components in different views. Some nonlimiting examples are illustrated in the figures of the accompanying drawings in which:

FIG. 1A is an illustration of a solid-state nanopore chip in the absence of a particle.

FIG. 1B is an illustration of the solid-state nanopore chip of FIG. 1A showing how the passage of a particle through the nanopore is detected as a current pulse.

FIG. 1C is a Transmission Electron Microscope (TEM) image of a 20 nm thickness SiNₓ nanopore.

FIG. 1D is a diagram of a sample nanopore detection instrument including the solid-state nanopore chip of FIG. 1A, a fluidic cell, and a portable nanopore reader.

FIG. 2 is a diagram illustrating a sample embodiment of an ultrathin solid-state nanopore chip fabrication procedure for producing the nanopore chip used with the configuration of FIG. 1D to detect diethyl phthalate (DEP) and other analytes in water.

FIG. 3A is a schematic of the analyte measurement setup and cross-sectional view of a glass chip and the SiNₓ nanopore in a sample configuration.

FIG. 3B is a TEM image of a 1.9 nm-diameter nanopore used for DEP translocation measurement in a sample configuration.

FIG. 4 is a diagram illustrating DEP detection using ultrathin and ultrasmall SiNₓ nanopores in a sample configuration.

FIG. 5A is a real-time current versus time trace showing a flat control current signal baseline of a 5 nm-thick, 1.5 nm-diameter SiNₓ nanopore without any molecules added.

FIG. 5B is a real-time current versus time trace showing the translocation of DEP molecules through the same pore of FIG. 5A.

FIG. 5C is a diagram of representative DEP translocation events extracted from FIG. 5B.

FIG. 6 is a histogram of blocked currents for measured translocation events measured in FIG. 5.

### DETAILED DESCRIPTION

To provide a fast, simple and reliable way of identifying unwanted constituents present in water systems such as a water system aboard the International Space Station (ISS) and potentially other spacecraft (e.g., Artemis Gateway Outpost), a robust, portable and easy-to-use sensor system based on solid-state nanopore technology is described. In the sample application of a water monitoring system for use in a spacecraft such as the ISS, it is observed that such spacecraft provide a highly controlled, stable, and isolated environment. The current water monitoring capability in the ISS, for example, is limited to electrical conductivity (for inorganics), total organic carbon (for organics), and selected ions of iodine and silver (residual disinfectants). To detect other analytes in water, samples must be brought down to earth. The water quality and safety is designed into process hardware and a monitoring system (e.g., sensors) can be imparted to make sure that hardware is operating normally. If there is a trigger indicating something is wrong, that means the quality could be degraded and the crew would have to do the troubleshooting. Thus, there is a strong demand for sensors that need little calibration and that are simple to use and do not require significant crew time. The single-molecule nanopore sensor described herein features minimal standard calibration and a quick turnaround time and may be operated by non-experts for monitoring water quality in the ISS and other crewed spacecrafts, as well as in other water quality monitoring applications on earth.

The solid-state nanopores described herein present an inherent single-molecule sensor system that works on the principle of pore occlusion by the molecule which then can be registered as a change of the ionic current. Each analyte establishes its unique signal upon passing through the nanopore of tailored characteristics. As described in commonly owned co-pending U.S. patent application Ser. No. 18/220,521 (Docket No.: Goeppert-001US1), entitled “Water Monitoring With Solid State Nanopores,” the detection of mercury and lead ions is possible using 2-5 nm-diameter and 20-nm thick nanopores at concentrations down to 5 nM and 0.5 nM, respectively, both of which are below both EPA requirements and Space Water Exposure Guidelines. As described further below, the sensor system described therein has been further enhanced and successfully used to detect diethyl phthalate (DEP), a small organic analyte (˜1 nm) that is desirable to measure for water monitoring purposes. The techniques described herein permit DEP to be measured without tedious sample preparation and heavy use of organic solvents that are required in the typical mass spectroscopic methods on earth. This direct detection of the naked molecule is enabled by ultrathin (˜<5 nm) and ultrasmall (˜1.5 nm) solid-state nanopores that allow the detection of low-concentration analytes in water and is thus a promising tool for a miniaturized analytical laboratory for future space missions.

A miniature analytical sensing instrument based on solid-state nanopores is described herein to enable spacecraft water monitoring on long-duration manned missions, such as transit to, and surface exploration of, the moon and Mars. One of the most important functions of life-support systems for manned space missions is ensuring the quality of potable water to protect astronaut health. This requires detection and characterization of both organic and inorganic contaminants, a process that currently requires numerous types of equipment. The instrument described herein is a compact, low-energy, simple-to-use, sensitive, and specific analytical instrument capable of detection and quantification of multiple organic and inorganic contaminants in water. This analytical instrument addresses the need for more comprehensive water monitoring capabilities for long-duration manned space missions that will require recycling and recovery of nearly every drop of water which places great emphasis on the ability of the water monitoring instrument to detect a wide range of organic and inorganic contaminants. In addition to detection of a wide range of contaminants, the system described herein possesses the flexibility to adapt to new and emerging potential contaminants that may arise.

The aforementioned commonly owned co-pending application demonstrated the technical feasibility of using solid-state nanopore sensors to detect low concentrations of select inorganic heavy metal ions [lead (Pb²⁺) and mercury (Hg²⁺)] using 2-5 nm-diameter and 20-nm thick SiNₓ nanopores. The sensor platform described therein has been expanded to provide improved dynamic range, and lower detection limits in a single analytical instrument. Together with the sensor platform described therein, the sensor system described herein enables miniature analytical systems to measure metal ions and organic constituents in potable water.

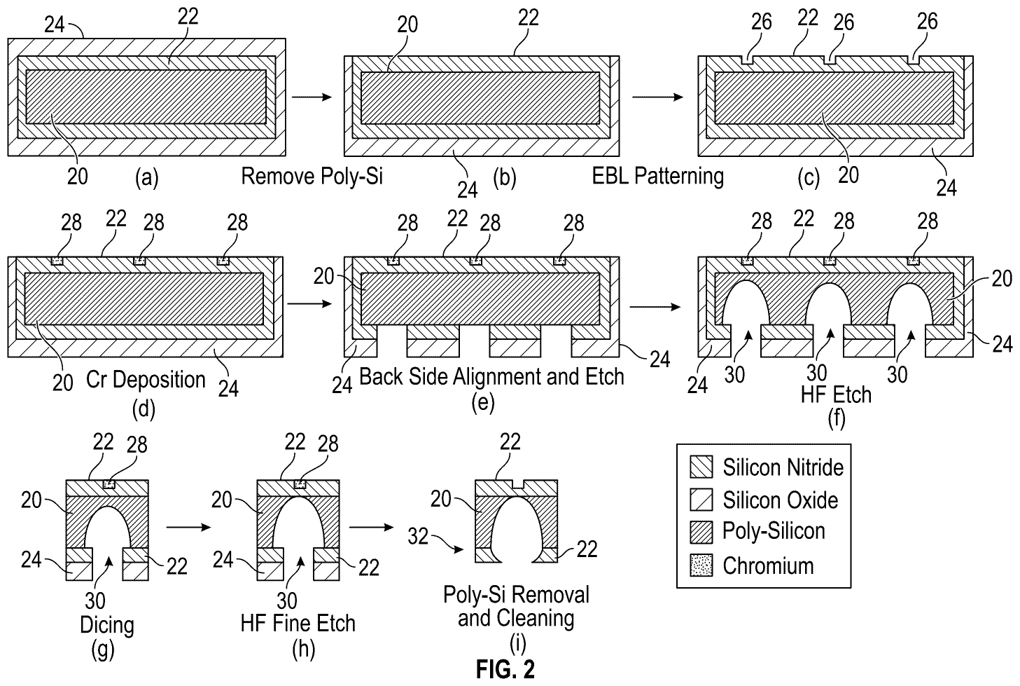

A detailed description of the methodology and sensor system for measuring organic species such as DEP in water will be described with reference to FIGS. 2-6. Although this description provides a detailed description of possible implementations, it should be noted that these details are intended to be exemplary and in no way delimit the scope of the inventive subject matter.

As will be described below with respect to FIG. 2, the nanopore sensor may be formed by starting with a 250 μm thick fused silicon oxide (silica) glass wafer with 50 nm thick silicon nitride (SiNₓ) and 125 nm of poly-silicon. The poly-silicon layer is removed from one side of the fused silica wafer by wet etching with potassium hydroxide (KOH). Electron beam lithography (EBL) may be used to pattern an array of 100×100 nm² squares and alignment marks onto this side of the wafer using a bilayer polymethyl methacrylate (PMMA) process. Reactive ion etching (RIE) may be used to thin the SiNₓ membrane under this pattern from 50 nm to 15 nm (or lower if desired). Chromium (Cr) may be deposited by pressure vapor deposition (PVD) and the lift-off of the PMMA is achieved by soaking the whole wafer in acetone. Photoresist is used to pattern on the opposite side of the wafer, and back-side alignment to Cr alignment marks may be used to pattern 10 μm square marks opposite to the thinned regions. The back side of the wafer is etched by hydrogen fluoride (HF) until the free-standing SiNₓ membranes are created. Poly-silicon on this side is removed with KOH wet etch. The Cr is removed with a chromium etchant. Wafers are cleaned in water and sent out for dicing into 5×5 mm² chips. The SiNₓ membrane on the chips can be further RIE thinned to less than 5 nm. The resulting chips are non-toxic and can be recycled to reduce the environmental risks.

Individual chips are processed to create nanopores in free-standing membranes by ablation with a transmission electron microscope. The chips are treated with UV-Ozone for cleaning and stored in air or a water/ethanol mixture. The resulting chips are measured using an Elements™ NPR amplifier and placed in a fluidic cell with potassium chloride (KCl) solution. A voltage is applied using Ag/AgCl electrodes. The NR-constrained bandwidth (Bmax) of this configuration was observed to obey the equation

$${B}_{max} ∝ {( \frac{Δ I}{{v}_{n} ∈ {C}_{i}} )}^{2 / 3} ,$$

where νₙ is the voltage thermal noise floor of the amplifier and ΣCᵢ is the net capacitance at the input of the amplifier, composed of contributions from the pore (C_{chip}), and the measurement electronics (Cₐₘₚ).

### Materials and Methods

### Materials and Solution Chemistry

Diethyl phthalate (DEP) was purchased from Sigma Aldrich (Merck, Rahway, NJ), and all chemicals were prepared using Milli-Q (Millipore, Billerica, MA) water with a resistivity of 18.2 MΩ cm⁻¹. Experiments were conducted in 0.5 M KCl at pH 5. All stock solutions were stored at 4° C. and used within one week of initial preparation.

### Ultrathin (˜<5 nm) SiNₓ Chips Fabrication

Nanopores were fabricated in ˜<5 nm low-stress SiNₓ membrane suspended on low-noise fused-silica (glass) chips. These glass chips were 5×5 mm², only 250 μm thick, and yielded significantly reduced capacitance (as low as <1 pF), making them excellent for high-bandwidth applications. The nanopore chip fabrication procedure is shown in FIG. 2.

As shown in FIG. 2, the fabrication procedure starts at (a) with a 250 μm thick fused silicon oxide glass wafer 20 with 50 nm of low stress SiNₓ 22 and 125 nm of poly-silicon 24 deposited thereon. At (b), the top poly-silicon layer 24 is removed and small 100×100 nm² squares 26 are patterned using electron-beam lithography (EBL) at (c). The squares 26 are thinned to 15 nm or less using trichloromethane (CHF₃) and O₂ reactive ion etching (RIE). These small regions 26 then may be coated at (d) with chromium (Cr) 28 via pressure vapor deposition (PVD). The Cr coating 28 allows alignment marks to be easily seen that may be used to provide pattern on the back side of the fused silica wafer using RIE etching. The HF bulk etching as shown at (f) is to remove the silicon oxide and almost expose the free-standing SiNₓ windows 30. After dicing into 5×5 nm² chips at (g), potassium hydroxide (KOH) etching was applied at (h) to remove poly-silicon 24. Fine HF etching was applied to remove the residual silicon oxide and expose the 50 μm diameter SiNₓ windows 30. The chromium is removed at (i) via a chromium etchant (e.g., Sigma Aldrich, MO) to create free standing SiNₓ membranes 22. The membrane 22 on the chip 32 was further thinned to ˜<5 nm using CHF₃ and O₂ RIE. Since the membrane 22 is locally thinned, it is robust and can be further thinned to sub-5 nm (e.g., 1.5 nm-2.0 nm), giving high spatial sensitivity.

### Nanopore Fabrication, Cleaning and Characterization

Nanopores are drilled in the thinnest area of the free-standing SiNₓ membranes 32 with a 200 keV focused electron beam in a JEOL 2010F transmission electron microscope (TEM). The TEM diameters of the pore, d_{TEM}, are measured right after drilling in vacuum. After drilling and prior to ionic measurement, pores are cleaned by UV/ozone treatment to remove organic contaminants and aid pore wetting. The UV/ozone treatment is done by exposing the chip under the UV/ozone lamp (6W, emission wavelengths of 254 and 185 nm) held about 5 cm above the chips for 1 hour 40 minutes on each side. After cleaning, the protonation/deprotonation of silanol and amine groups in the electrolyte solution make the surface negatively or positively charged and the surface properties of SiNₓ dependent on its preparation process. The pores then are either inserted in ionic solution for measurement or kept in air or a water/ethanol mixture.

Nanopore diameters can change once the chip is taken out of the TEM chamber and used for ionic solution measurements. Pore diameters during the ionic measurement can be estimated using Equation (1) (assuming membrane thickness t=5 nm), which provides nanopore conductance (G) as:

$$G = \frac{I}{{V}_{bias}} = {σ ( \frac{4 t}{π {d}_{calc}^{2}} + \frac{1}{{d}_{calc}} )}^{- 1} \qquad Equation ( 1 )$$

where d_{calc} is the calculated diameter of the nanopore, I is the open pore current without adding any analyte, V_{bias} is the applied transmembrane voltage, t is the thickness of the membrane, and σ is the ionic solution conductivity (σ˜12 S/m for 1 M KCl at room temperature).

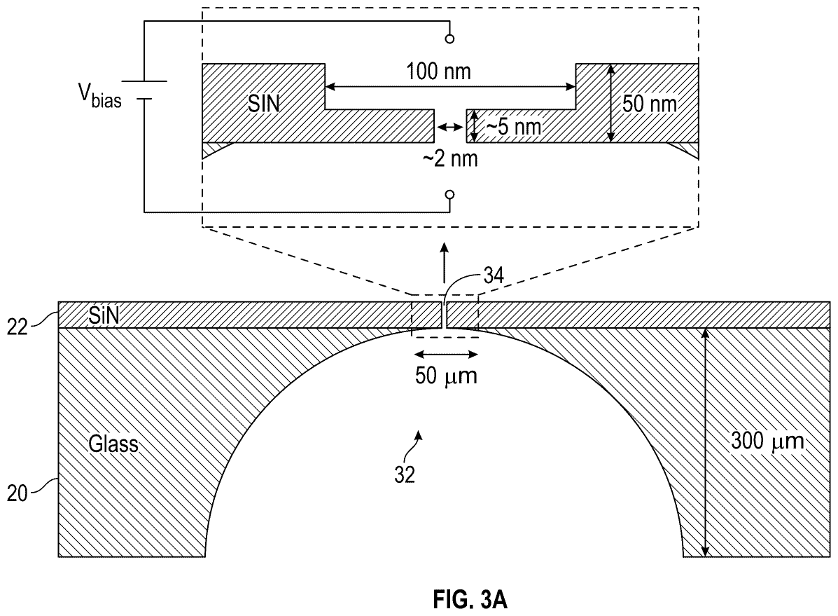

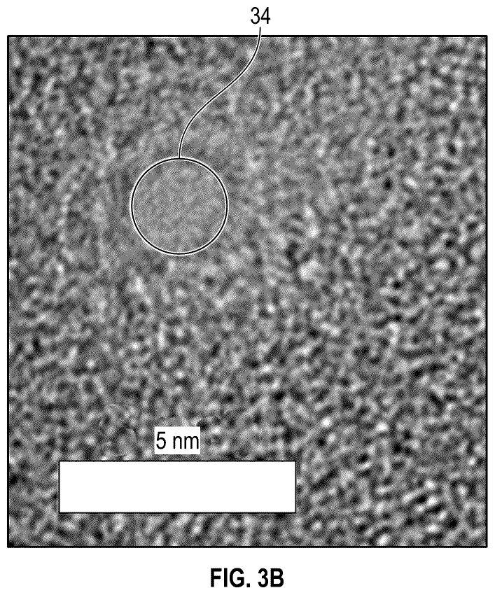

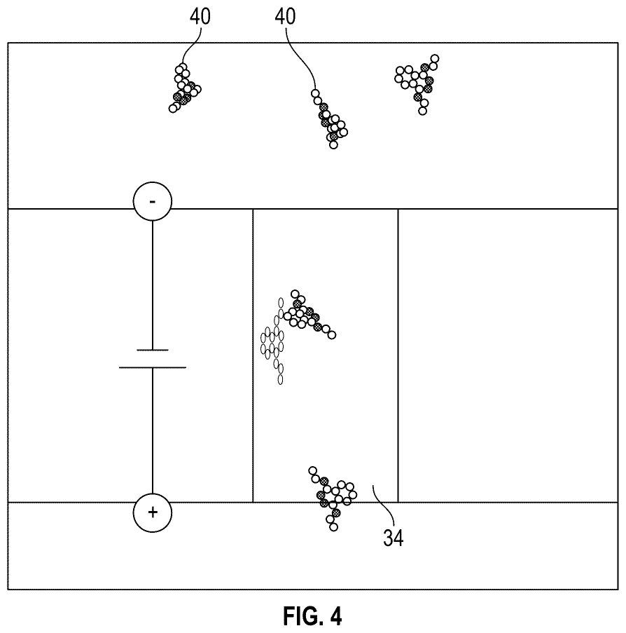

The nanopore chip 32 with TEM diameter of ˜1.9 nm (FIG. 3) was used in a DEP measurement. FIG. 3A is a schematic of the measurement setup and cross-sectional view of a glass chip and the SiNₓ nanopore 34, while FIG. 3B is a TEM image of a 1.9 nm-diameter nanopore used for DEP translocation measurement. In the example of FIG. 3A, the nanopore chip 32 has a thickness of ˜5 nm at the pore 34, which has a diameter of ˜2 nm (e.g., ˜1.9 nm). Transmembrane voltage V_{bias} is applied to drive the analytes through the pore 34.

### Nanopore Measurements for DEP Detection

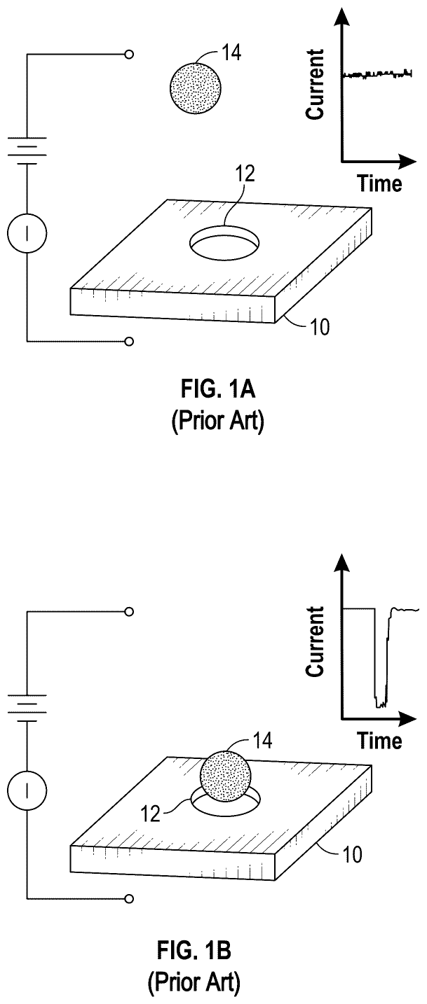

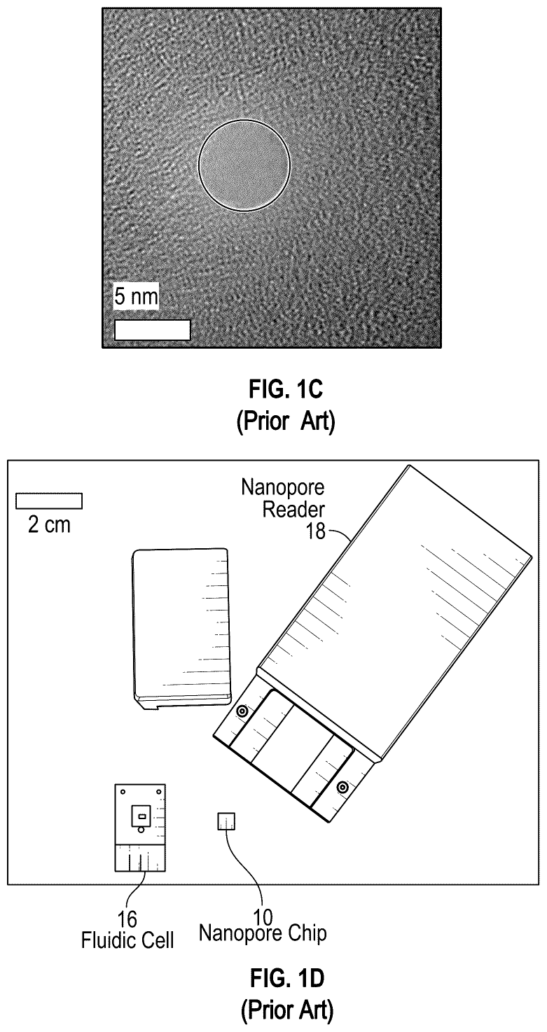

For testing, the nanopore chips 32 were assembled in a custom-designed fluidic cell 16 (FIG. 1D). DEP samples were measured as they were driven through the pores 34 at voltage V_{bias} of +100 to +400 mV. Buffer solutions containing DEP were added to the cis-side of the nanopore 34 for the first control measurement. The samples were prepared in buffer containing 0.5 M KCl at pH 5. The trans-chamber was filled with the same buffer. The passage of DEP across the nano pore 34 caused a reduction of the current. The real time analyses of the electrical spikes provided a reference signal for subsequent experiments.

A portable nanopore reader 18 with a current amplifier (FIG. 1D) was used to record translocation events of molecules one at a time. The portable nanopore reader 18 supports a sampling rate up to 200 k samples/second. Data were analyzed using pCLAMP 10.3 (Molecular Devices, CA), with which the characteristic dwell time (duration of each translocation event) and the mean current blockade (magnitude of the ionic current reduction) were extracted. Data were further analyzed using OriginLab (OriginLab, MA). For each set of measurements, data were collected using the same nanopore 34 to minimize possible influence from pore-to-pore variance unless significant pore expansion or clogging was observed. The nanopore chips 32 were rinsed with deionized water and isopropanol between each measurement to eliminate cross-contamination and to restore a flat, baseline signal. The experiment was repeated using two nanopore chips for each experiment to confirm the trends reported.

Diethyl phthalate (DEP) was detected by the solid state nanopore chip so designed. DEP 40 was directly detected without the aid from any molecular carriers using ultrathin and ultrasmall SiNₓ nanopores 34 as illustrated in FIG. 4. It will be appreciated by those skilled in the art that DEP appears as a clear, colorless liquid without significant odor used as a plasticizer for fabricating flexible materials and products. It belongs to the phthalate group and has been found to accumulate in the recycled water, cabin humidity condensate (the principal source of potable water) and air filters in space shuttles and aboard the international space station (ISS) and are potentially of concern to crew health. The molecular structure of DEP is as follows:

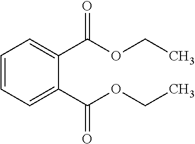 [SMILES: CCOC(=O)c1ccccc1C(=O)OCC]

Some possible sources of DEP in the ISS include leaching from the plastic materials to food and drink, contaminants from crew activities released into the cabin air, from payload experiments and onboard utility chemicals and similar materials. Dibutyl phthalate (DBP), another organic compound of similar structure to DEP, is identified in the Space Water Exposure Guidelines with a maximum contaminant level (MCL) of 40 mg/L.

To detect DEP and other small (e.g., <1 nm diameter) organic analytes in water samples, an ultrathin (˜5 nm) nanopore 34 with an ultrasmall diameter of only ˜1.5 nm was used. This diameter is slightly larger and allows the translocation of one single DEP molecule each time, whose dimension is approximately 0.9 nm×0.6 nm. Using this nanopore 34, the translocation of DEP at the single-molecule resolution was successfully detected directly without the aid from any molecular probes, as compared to the aptamer method for ion detection described in commonly owned and copending U.S. application Ser. No. 18/220,521 (Docket No.: Goeppert-001US1) entitled “Water Monitoring With Solid State Nanopores.” The electrolyte solution used for these measurements was 0.5 M KCl at pH 5. DEP was added at a concentration of 1 g/L to the flow cell and a bias voltage of +300 mV was applied in order to drive the DEP through the nanopore 34. This concentration of DEP was chosen for test because the Space Water Exposure Guidelines have regulated similar or even higher daily potential exposure concentrations of 1.2 g/L and 1.8 g/L for its homogeneous compounds of di-n-butyl phthalate and di(2-ethylhexyl) phthalate, respectively.

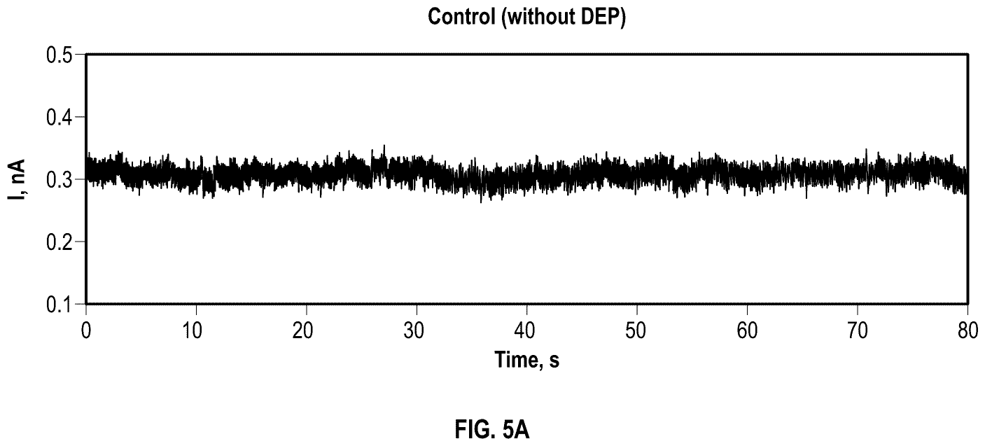

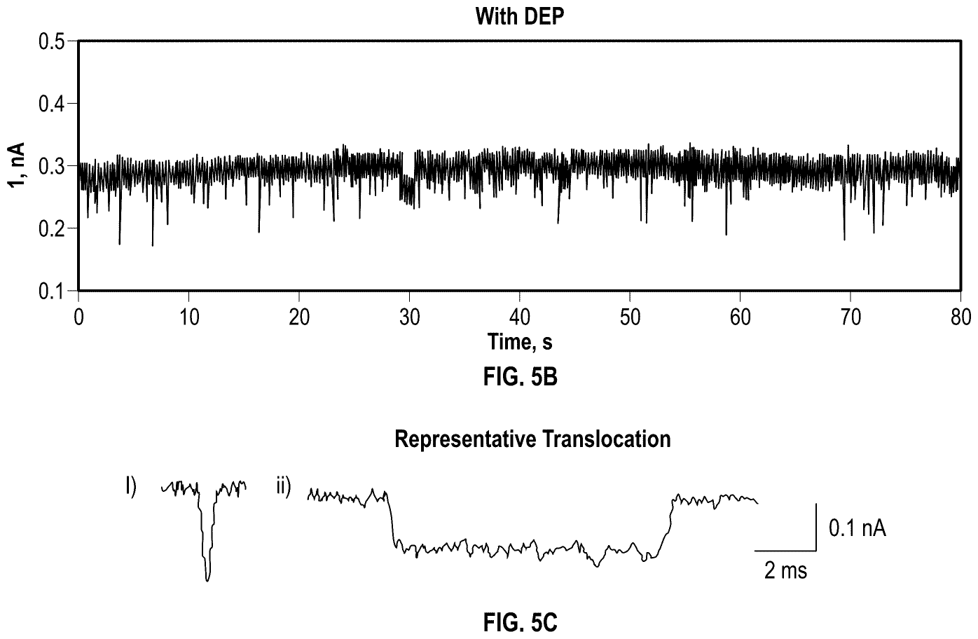

FIG. 5A is a real-time current versus time trace showing a flat control current signal baseline of a 5 nm-thick, 1.5 nm-diameter SiNₓ nanopore without any molecules added. FIG. 5B is a real-time current versus time trace showing the translocation of DEP molecules through the same pore of FIG. 5A. Data were collected at +300 mV with nanopore reader (FIG. 1) at measurement bandwidth of 20 kHz. The calculated root-mean-square (rms) noise of the trace, I_{BASELINE}^{RMS} is about 0.01 nA. Event recognition was performed with simple thresholding at multiples of σ=I_{BASELINE}^{RMS} and a thresholding of 5σ was chosen to reduce the false events during data recording. FIG. 5C is a diagram of representative DEP translocation events extracted from FIG. 5B.

The measured negative current spikes in FIG. 5 are consistent in that DEP are negatively charged molecules. The sampling rate is at 20 kHz. As shown in FIG. 5B, the current was observed to drop sharply as the DEP pass through the ultrasmall nanopore 34. Translocation events are not observed before the addition of DEP (FIG. 5A). FIG. 5C shows representative translocation events with durations as short as 0.2 ms (i) and as long as 6 ms (ii). The longer event could be the temporary sticking of DEP to the inside wall while passing through the nanopore 34. Given a bandwidth-limitation of 20 kHz in this measurement, it is possible that duration times in translocation measurements are skewed by bandwidth limitations. This has been documented, for example in DNA translocation measurements where 10 MHz bandwidth measurements yielded by one order of magnitude shorter characteristic dwell times compared to ˜1 MHz (˜10 ns/nucleotide). To detect molecules that pass through the nanopore 34 in a shorter time (less than about 200 s in this case), and also to have minimal distortions of the current blockade for slightly longer events, a higher bandwidth amplifier may be used (e.g., 1 MHz or 10 MHz amplifier) to achieve better temporal resolution.

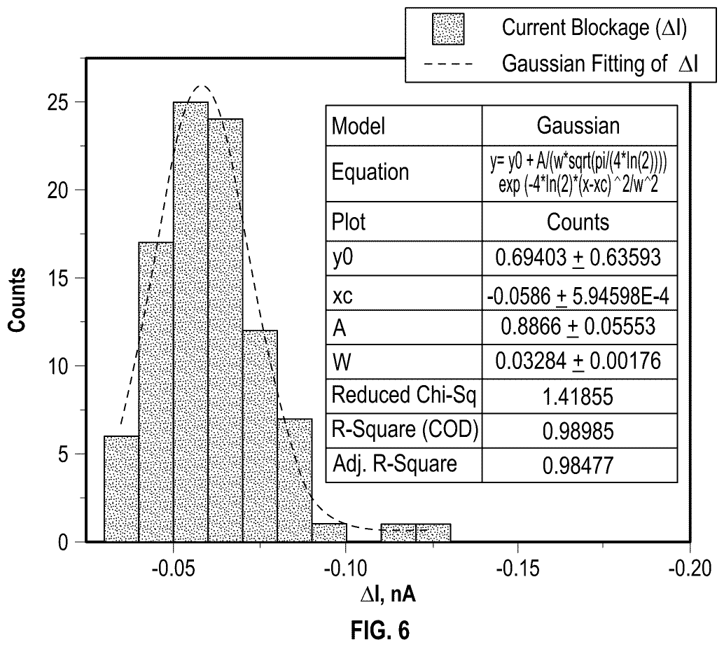

FIG. 6 is a histogram of the measured current blockade signal, ΔI, for measured translocation events measured in FIG. 5. Here, ΔI is defined as ΔI=I−Iₒₚₑₙ, where I is the mean pore current during DEP translocation and Iₒₚₑₙ is the mean pore current 1 ms before DEP entry. The histogram in FIG. 6 can be fit with a Gaussian with mean ΔI values of approximately −0.06 nA. Using Equation (1), the average diameter, D, of the translocating negative molecule can be estimated to be 0.7 nm. This is consistent with a DEP molecule which passes the nanopore in a linear, single-molecule translocation pattern.

Thus, diethyl phthalate (DEP) may be successfully detected without tedious sample preparation and heavy use of organic solvents which are required in the typical mass spectroscopic methods. This direct detection of the naked molecule is enabled by ultrathin (<5 nm) and ultrasmall (˜1.5 nm) SiNₓ nanopores produced using the fabrication techniques described herein. The fabrication techniques for fabricating the ultrathin (<5 nm) and ultrasmall (˜1.5 nm) SiNₓ nanopores allow for hundreds of chips to be patterned at a time using Electron Beam Lithography without needing an alignment step. High signal-to-noise nanopore chips may be manufactured at the wafer scale while overcoming the time consuming alignment in Electron Beam Lithography to free standing membranes in the prior art. Fused-silica chips provide low capacitance (˜2 pF), and local thinning provides robust silicon nitride membranes as thin as 5 nm. The resulting ultra-low noise locally thinned nanopore chips can be used as stochastic sensors.

It will be appreciated that solid state nanopore sensors with nanopores larger than 2 nm may be used with the resultant loss in sensitivity. For example, the 2.0-5.0 nm diameter nanopores described in commonly owned and copending U.S. application Ser. No. 18/220,521 (Docket No.: Goeppert-001US1) entitled “Water Monitoring With Solid State Nanopores” may also be used for direct detection of certain organic analytes such as DEP and dibutyl phthalate (DBP).

It will be understood by one skilled in the art that this disclosure is not limited in its application to the details of construction and the arrangement of components set forth in the description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings.

Although the present disclosure has been described with reference to specific features and embodiments thereof, it is evident that various modifications and combinations can be made thereto without departing from the scope of the disclosure. The specification and drawings are, accordingly, to be regarded simply as an illustration of the disclosure as defined by the appended claims, and are contemplated to cover any and all modifications, variations, combinations or equivalents that fall within the scope of the present disclosure.

What is claimed is:

1. A method of sensing an organic analyte in a fluid, comprising:
inserting an ultrathin solid-state nanopore chip having at least one nanopore of less than 5 nm diameter into a fluidic cell;
providing a fluid for testing for the organic analyte, the organic analyte having a diameter of approximately 1 nm or less; and
measuring concentration of the organic analyte as the fluid passes through the at least one nanopore of the solid-state nanopore chip.

2. The method of claim 1, wherein the solid-state nanopore chip comprises at least one silicon nitride (SiNₓ) nanopore having a thickness of approximately 5 nm at the at least one SiNₓ nanopore.

3. The method of claim 1, wherein the at least one nanopore has a diameter of approximately 2 nm.

4. The method of claim 3, wherein the at least one nanopore has a diameter of approximately 1.9 nm.

5. The method of claim 4, wherein the at least one nanopore has a diameter of approximately 1.5 nm.

6. The method of claim 1, wherein the organic analyte comprises at least one of diethyl phthalate (DEP).

7. The method of claim 1, wherein measuring concentration of the organic analyte comprises recording translocation events of organic analytes one at a time using a nanopore reader as the organic analytes translocate through the at least one nanopore.

8. The method of claim 1, wherein the fluid is water.

9. A water monitoring system adapted to sense an organic analyte in water, comprising:
a fluidic cell that holds water for testing;
a solid-state nanopore chip having at least one nanopore of less than 5 nm diameter disposed in the fluidic cell for testing the water for the organic analyte, the organic analyte having a diameter of approximately 1 nm or less; and
a nanopore reader that measures concentration of the organic analyte as the water passes through the at least one nanopore of the solid-state nanopore chip.

10. The water monitoring system of claim 9, wherein the fluidic cell is disposed on a spacecraft.

11. The water monitoring system of claim 9, wherein the solid-state nanopore chip comprises at least one silicon nitride (SiNₓ) nanopore having a thickness of approximately 5 nm at the at least one SiNₓ nanopore.

12. The water monitoring system of claim 9, wherein the at least one nanopore has a diameter of approximately 2 nm.

13. The water monitoring system of claim 12, wherein the at least one nanopore has a diameter of approximately 1.9 nm.

14. The water monitoring system of claim 13, wherein the at least one nanopore has a diameter of approximately 1.5 nm.

15. The water monitoring system of claim 9, wherein the organic analyte comprises at least one of diethyl phthalate (DEP).

16. The water monitoring system of claim 9, wherein the nanopore reader records translocation events of organic analytes one at a time as the organic analytes translocate through the at least one nanopore.

17. The water monitoring system of claim 9, wherein the solid-state nanopore chip is fabricated from a fused silicon oxide glass wafer coated on a first side with a low stress SiNₓ and poly-silicon by removing a top poly-silicon layer from the first side by wet etching with potassium hydroxide (KOH) and patterning small regions using electron-beam lithography (EBL) that are thinned to 15 nm or less using CHF₃ and O₂ reactive ion etching (RIE), using photolithography and RIE on a second side of the wafer and etching SiNₓ using hydrofluoric acid (HF) to create SiNₓ windows, dicing the SiNₓ windows into approximately 5×5 nm² chips, and applying KOH etching to remove poly-silicon and fine HF etching to expose the SiNₓ windows.

18. A method of fabricating a solid-state nanopore chip having at least one nanopore of less than 5 nm diameter, comprising:
coating a fused silicon oxide glass wafer on a first side with a low stress SiNₓ and poly-silicon;
removing a top poly-silicon layer from the first side by wet etching with potassium hydroxide (KOH);
patterning small regions using electron-beam lithography (EBL) that are thinned to 15 nm or less using CHF₃ and O₂ reactive ion etching (RIE);
using photolithography and RIE on a second side of the wafer and etching the SiNₓ using hydrofluoric acid (HF) to create SiNₓ windows;
dicing the SiNₓ windows into approximately 5×5 nm² chips; and
applying KOH etching to remove poly-silicon and fine HF etching to expose the SiNₓ windows.

19. The method of claim 18, further comprising thinning the solid-state nanopore chip to a diameter of approximately 5 nm using CHF₃ and O₂ RIE.

20. The method of claim 19, further comprising thinning the solid-state nanopore chip to a diameter of approximately 1.5-2.0 nm using CHF₃ and O₂ RIE.
